(19) United States
(12) Patent Application Publication
(10) Pub. No.: US 2023/0372089 A1
(43) Pub. Date: Nov. 23, 2023

(54) SYSTEMS AND METHODS FOR OPTIMIZING BLOOD FLOW

(71) Applicant: 4C Medical Technologies, Inc., Maple Grove, MN (US)

(72) Inventor: Saravana B. Kumar, Minnetonka, MN (US)

(21) Appl. No.: 18/200,691

(22) Filed: May 23, 2023

Related U.S. Application Data
(60) Provisional application No. 63/344,713, filed on May 23, 2022.

(51) Int. Cl. A61F 2/24

(52) U.S. Cl. CPC A61F 2/2418

(57) ABSTRACT

A collapsible and expandable prosthetic heart valve stent is provided that may be adapted to provide a one-way blood flow therethrough that targets blood flow at the posterior lateral side of a ventricle to enable a natural blood flow to persist post-implant.

### CROSS REFERENCE TO RELATED APPLICATIONS

This application is a nonprovisional of U.S. Provisional Application No. 63/344,713 filed on May 23, 2022, which is hereby incorporated herein by reference in its entirety.

### BACKGROUND

The human heart comprises four chambers and four heart valves that assist in the forward (antegrade) flow of blood through the heart. The chambers include the left atrium, left ventricle, right atrium, and right ventricle. The four heart valves include the mitral valve, the tricuspid valve, the aortic valve, and the pulmonary valve. See generally FIG. 1.

The mitral valve is located between the left atrium and left ventricle and helps control the flow of blood from the left atrium to the left ventricle by acting as a one-way valve to prevent backflow into the left atrium. Similarly, the tricuspid valve is located between the right atrium and the right ventricle, while the aortic valve and the pulmonary valve are semilunar valves located in arteries flowing blood away from the heart. The valves are all one-way valves, with leaflets that open to allow forward (antegrade) blood flow. The normally functioning valve leaflets close under the pressure exerted by reverse blood to prevent backflow (retrograde) of the blood into the chamber it just flowed out of. For example, the mitral valve when working properly provides a one-way valving between the left atrium and the left ventricle, opening to allow antegrade flow from the left atrium to the left ventricle and closing to prevent retrograde flow from the left ventricle into the left atrium. This retrograde flow, when present, is known as mitral regurgitation or mitral valve regurgitation. As is shown, normal blood flow proceeds through the mitral valve from the left atrium to the left ventricle impinging on the posterior lateral side of the left ventricle (as opposed to the septal side). This natural flow takes advantage of the left ventricular anatomy so that the flow is further directed downward within the left ventricle and then upward toward the aortic valve and the left ventricular outflow tract (LVOT) and the associated aortic root, and ultimately into the ascending aorta as shown.

FIG. 2 illustrates the relationship between the left atrium, annulus, chordae tendineae and the left ventricle relative to the mitral valve leaflets. As is shown, the upper surface of the annulus forms at least a portion of the floor or lower surface of the left atrial chamber, so that for purposes of description herein, the upper surface of the annulus is defined as marking the lower boundary of the left atrial chamber.

Native heart valves may be, or become, dysfunctional for a variety of reasons and/or conditions including but not limited to disease, trauma, congenital malformations, and aging. These types of conditions may cause the valve structure to fail to close properly resulting in regurgitant retrograde flow of blood from the left ventricle to the left atrium in the case of a mitral valve failure.

FIG. 3 illustrates regurgitant blood flow with an exemplary dysfunctional mitral valve.

Mitral valve regurgitation is a specific problem resulting from a dysfunctional mitral valve that allows at least some retrograde blood flow back into the left atrium from the right atrium. In some cases, the dysfunction results from mitral valve leaflet(s) that prolapse up into the left atrial chamber, i.e., above the upper surface of the annulus instead of connecting or coapting to block retrograde flow. This backflow of blood places a burden on the left ventricle with a volume load that may lead to a series of left ventricular compensatory adaptations and adjustments, including remodeling of the ventricular chamber size and shape, that vary considerably during the prolonged clinical course of mitral regurgitation.

Regurgitation can be a problem with native heart valves generally, including tricuspid, aortic and pulmonary valves as well as mitral valves.

Native heart valves generally, e.g., mitral valves, therefore, may require functional repair and/or assistance, including a partial or complete replacement. Such intervention may take several forms including open heart surgery and open-heart implantation of a replacement heart valve.

Less invasive methods and devices for replacing a dysfunctional heart valve are also known and may involve percutaneous access and catheter-facilitated delivery of the replacement valve. Most of these solutions may involve a replacement heart valve attached to a structural support such as a stent, commonly known in the art, or other form of wire network designed to expand upon release from a delivery catheter. The self-expansion variants of the supporting stent assist in positioning the valve, and holding the expanded device in position, within the subject heart chamber or vessel. If the device is not properly positioned in the first positioning attempt, it may be recaptured and positionally adjusted. This recapturing process in the case of a fully, or even partially, expanded device may require re-collapsing the device to a point that allows the operator to retract the collapsed device back into a delivery sheath or catheter, adjust the inbound position for the device and then re-expand to the proper position by redeploying the positionally-adjusted device distally out of the delivery sheath or catheter. Collapsing the already expanded device may be difficult because the expanded stent or wire network is generally designed to achieve the expanded state which also resists contractive or collapsing forces.

Besides the open-heart surgical approach discussed above, gaining access to the valve of interest may be achieved percutaneously via one of at least the following known access routes: transapical; transfemoral; transatrial; and transseptal delivery techniques.

Generally, the art is focused on systems and methods that, using one of the above-described known access routes, allow a partial delivery of the collapsed valve device, wherein one end of the device may be released from a delivery sheath or catheter and expanded for an initial positioning followed by full release and expansion when proper positioning is achieved.

In addition, all known prosthetic heart valves are intended for full replacement of the native heart valve. Therefore, these replacement heart valves, and/or anchoring or tethering structures, physically may extend out of the left atrial chamber, in the case of mitral valves, and engage the inner annulus and/or valve leaflets, in many cases pinning the native leaflets against the walls of the inner annulus, thereby permanently eliminating all remaining functionality of the native valve and making the patient completely reliant on the replacement valve. In other cases, the anchoring structures may extend into the left ventricle and may anchor into the left ventricle wall tissue and/or the sub-annular surface at the top of the left ventricle. Others may comprise a presence in, or engagement with, a pulmonary artery.

Obviously, there will be cases when native valve has lost virtually complete functionality before the interventional implantation procedure. In this case the preferred solution may comprise an implant that does not extend outside of, e.g., the left atrium, and that functions to completely replace the native valve function. However, in many other cases, the native valve remains functional to an extent and may, or may not, continue to lose functionality after the implantation procedure.

Finally, known prosthetic cardiac valves may consist of two or three leaflets that are arranged to act as a one-way valve, permitting fluid flow therethrough in the antegrade direction while preventing retrograde flow. The native mitral valve is located retrosternally at the fourth costal cartilage, consisting of an anterior and posterior leaflet, chordae tendineae, papillary muscles, ventricular wall and annulus connected to the atria. Each native leaflet is supported by chordae tendineae that are attached to papillary muscles which become taut with each ventricular contraction preserving valvular competence. Both the anterior and posterior leaflets of the native valve are attached via primary, secondary and tertiary chordae to both the antero-lateral and posterior-medial papillary muscles. A disruption in either papillary muscle in the setting of myocardial injury, may result in dysfunction of either the anterior or posterior leaflet of the mitral valve. Other mechanisms may result in failure of one, or both of the native mitral leaflets. In the case of a single mitral valve leaflet failure, the regurgitation may take the form of a non-central, eccentric jet of blood back into the left atrium. Other leaflet failures may comprise a more centralized regurgitation jet. Known prosthetic valve replacements may generally comprise leaflets which are arranged to mimic the native valve structure, which may over time become susceptible to similar regurgitation outcomes.

It would be desirable, therefore, to provide apparatus and methods for remediating valve failure.

### BRIEF DESCRIPTION OF THE DRAWINGS

FIG. 1 illustrates certain features of the heart in cross-section.

FIG. 2 illustrates a perspective view of the left side of the heart.

FIG. 3 illustrates a cross-sectional view of the heart showing retrograde blood flow resulting from mitral valve regurgitation compared with normal blood flow.

FIG. 4 illustrates a cutaway view of normal blood flow from the left atrium to the aortic root.

FIG. 5 illustrates a cutaway view of modified blood flow from the left atrium to the aortic root resulting from prior art mitral valve implant device.

FIG. 6 illustrates a cutaway view of modified blood flow from the left atrium to the aortic root resulting from prior art mitral valve implant device.

FIG. 7 illustrates a cutaway view of modified blood flow from the left atrium to the aortic root resulting from prior art mitral valve implant device.

FIG. 8 illustrates a cutaway view of modified blood flow from the left atrium to the aortic root resulting from prior art mitral valve implant device.

FIG. 9 illustrates a perspective view of one embodiment of a collapsible stent frame with valve support according to the present invention.

FIG. 10 illustrates a perspective view of one embodiment of a collapsible stent frame with valve support according to the present invention.

FIG. 11 illustrates a partial cutaway view of one embodiment of a prosthetic mitral valve device of the present invention expanded and implanted into the left atrium, with resulting normal blood flow.

FIG. 12 illustrates a partial cutaway view of one embodiment of a prosthetic mitral valve device of the present invention expanded and implanted into the left atrium, with resulting normal blood flow.

### DESCRIPTION

The apparatus and methods may be related to delivery and implantation of a valve device that will function both as a supplemental or augmentation valve without damaging the native leaflets in order to retain native valve leaflet functionality as long as present. This apparatus and methods may also be fully capable of replacing the native function of a valve that slowly loses most or all of its functionality post-implantation of the prosthetic valve.

The apparatus and methods may extend the functionality of native valve leaflets in a supplement first and may replace when required structure as described herein. It may be highly advantageous in this regard to provide a structure that only engages prolapsing native leaflets at a point of coaptation, thus preventing prolapse.

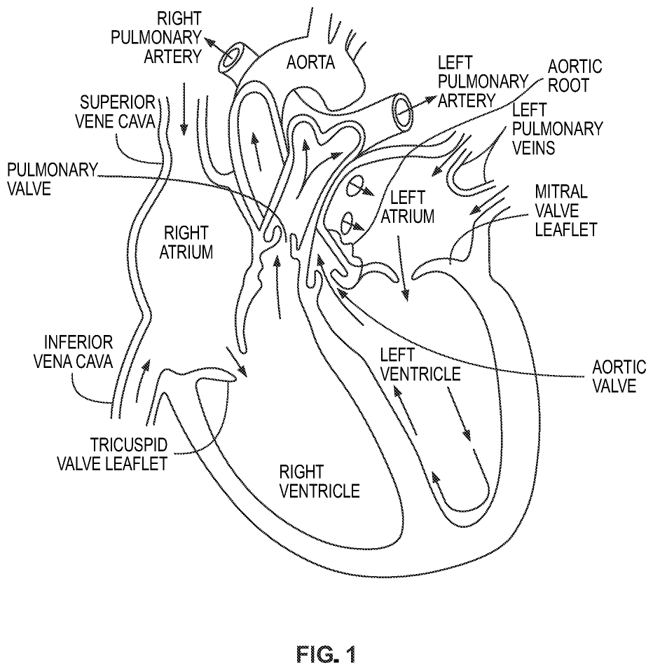

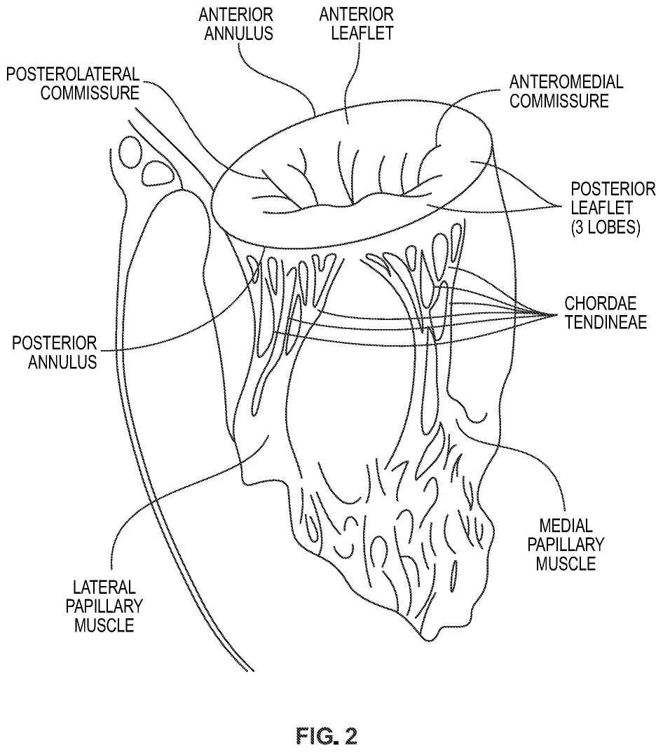

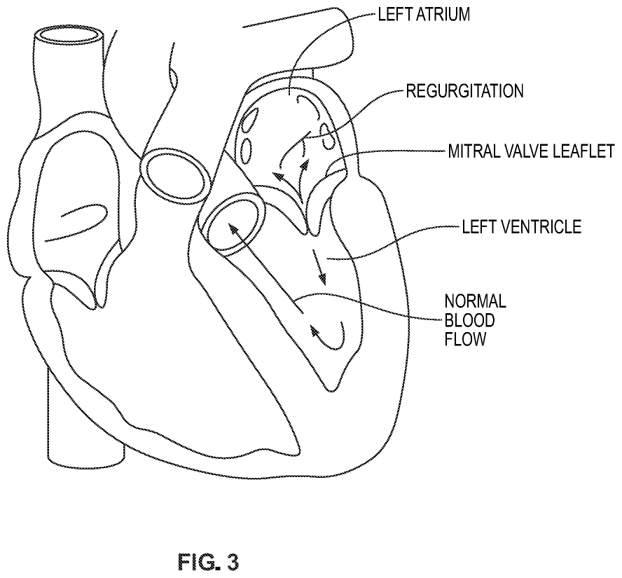

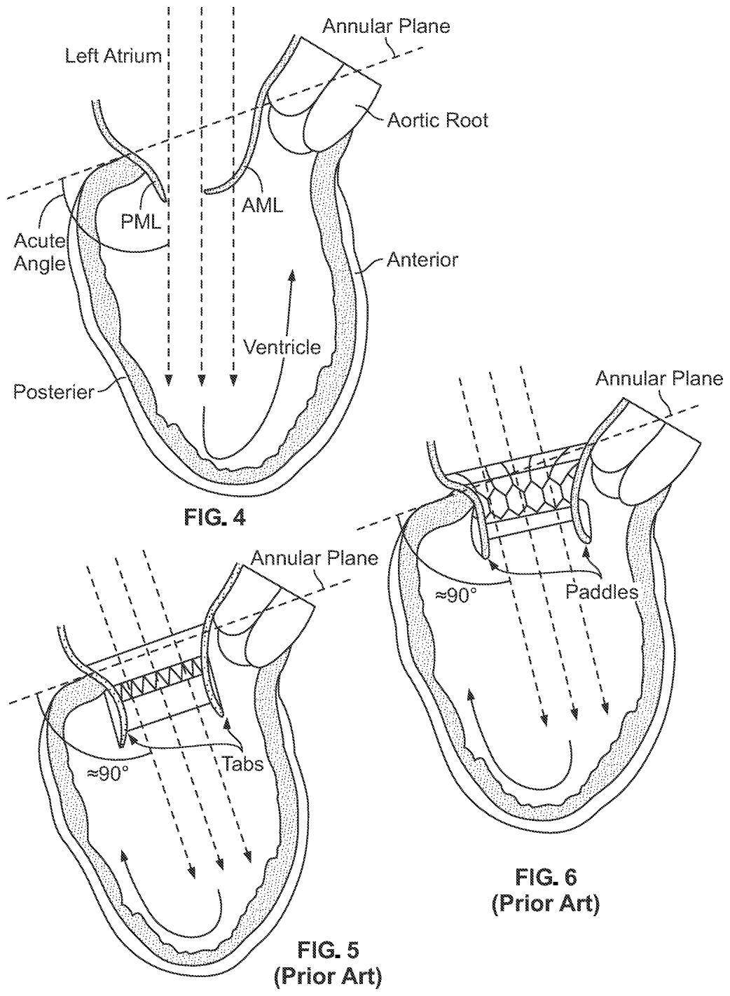

The apparatus and methods may supplement, preserve, or replace native mitral valve functionality. In this way, as generally shown in FIG. 1 and again in FIG. 4, a pattern of a natural flow of blood through the mitral valve may be achieved. The flow may continue through the left ventricle, LVOT, aortic root and aorta. A blood flow towards the posterior, lateral side of the left ventricle allows taking advantage of the anatomy of the left ventricle to transfer the fluid flow around the lower portion (or apical region) of the left ventricle and upward toward the LVOT and aorta. The flow may conserve energy by taking advantage of the anatomy and energy within the flowing blood and may help the left ventricle function efficiently.

As shown in FIGS. 5-8, implantation of known prosthetic mitral valve devices may alter the natural blood flow of FIGS. 1 and 4. As shown, in each case, the prosthetic mitral valve device may engage the native mitral valve leaflets. This may pin them or prevent them from moving to coapt.

FIG. 4 shows the natural mitral valve leaflets' role in directing the blood flow against the posterior lateral side of the left ventricle. The role includes the role of the posterior mitral leaflet (“PML”) and the anterior mitral leaflet (“AML”).

FIG. 5 shows an implanted device with tabs that may engage each of the native mitral valve leaflets, possibly preventing them from moving and coapting.

FIG. 6 shows an implanted device with paddle structures that similarly may engage and possibly prevent the native mitral valve leaflets from moving and/or coapting.

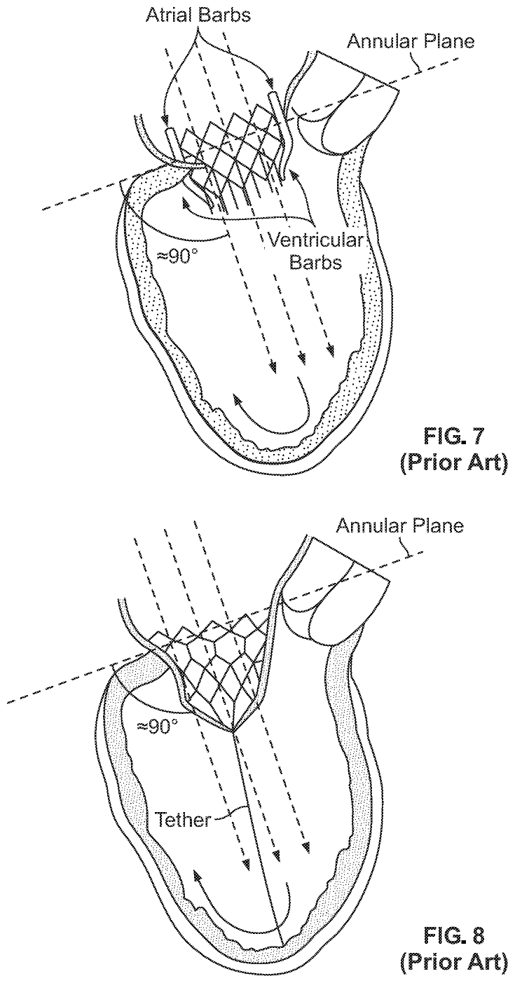

FIG. 7 shows an implanted device that extends downward into the left ventricle and may engage the native mitral valve leaflets, also possibly preventing the native leaflets from moving and/or coapting.

FIG. 8 shows an implanted device that extends downward to engage the native mitral valve leaflets and further comprises a tether that extends through the left ventricle, engaging the apical region. Also this device possibly prevents the native leaflets from moving and/or coapting.

In each of the illustrated cases of FIGS. 5-8, the implanted device may be oriented within, and aligned with, the annular throat or channel. As shown, this implantation orientation may result in a device that extends downstream to the ends of the native mitral valve leaflets. Because the annular throat or channel is slightly tilted downward in the posterior direction, the implanted altitude or position of the known implants is similarly tilted according to the annular throat or channel's anatomical tilt. Stated differently, the flow channels of each of the implanted devices of FIGS. 5-8 may all be substantially perpendicular to the annular plane, defined as a line drawn across the upper annular surface of the mitral valve and as illustrated.

FIGS. 5-8 show that these arrangements may produce a modified flow that is different from the natural blood flow. The modified blood flow is directed toward the septal side of the left ventricle, as opposed to the posterior side in a normal flow pattern. The flow of the blood may be, in accordance with the flow channel orientation in each of FIGS. 5-8, essentially perpendicular to the annular plane, which may cause the blood to impinge on the septal, as opposed to the posterior, side of the left ventricle. The natural blood flow of FIG. 4 shows an acute angle on the posterior side of the mitral valve, relative to the annular plane, while the modified blood flow of FIGS. 5-8 may provide a substantially 90-degree angle for the blood flow relative to the annular plane as shown.

As can be seen, the modified blood flow is required to move downward from the septal landing point, around the apical region (away from the LVOT and aortic root), where it must then find its way through the incoming flow to reach the LVOT and aorta. Essentially, the flow in the modified cases of FIGS. 5-8 may be required to move in a reverse direction through the left ventricle in order to reach the LVOT.

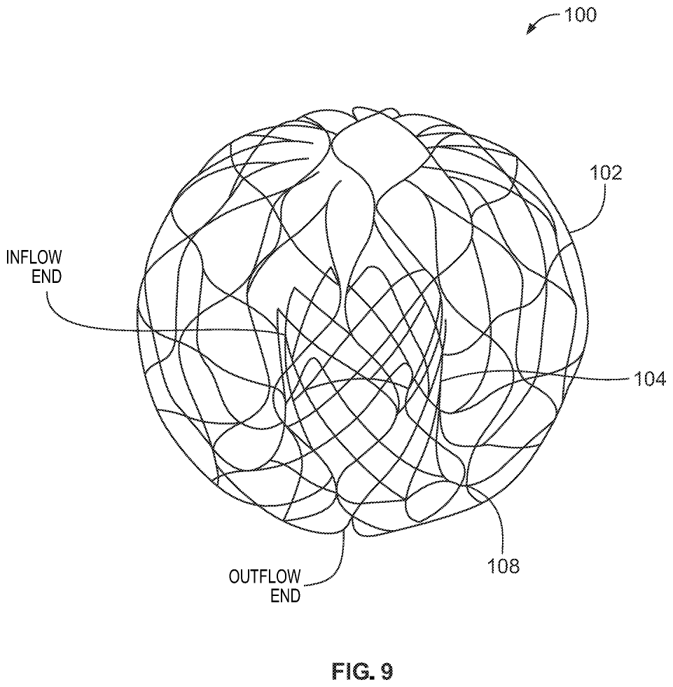

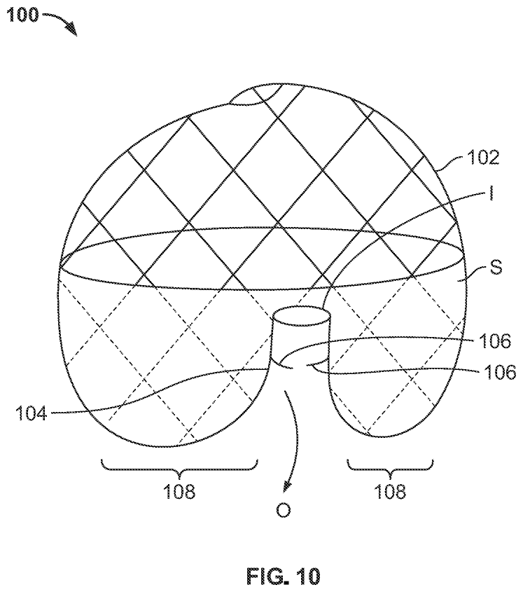

Turning now to FIGS. 9 and 10, an exemplary embodiment of an expandable and collapsible prosthetic heart valve stent 100 may include an outer section 102 that may generally look like a ball or an ellipse when undeformed and fully expanded. Stent 100 may include inner valve support section 104. Section 104 may be adapted to support and retain prosthetic valve leaflets 106. Leaflets 106 may be supported within inner valve support section 104. Leaflets 106 may be supported at a point located above the native annulus. Leaflets 106 may be spaced away from or above the native leaflets. Leaflets 106 may be positioned, relative to the native leaflets, in any suitable position. Inner valve support 104 may be cylindrical. Inner valve support 104 may be conical or partially conical. A conical or partially conical valve support 104 may have a diameter at outflow end O that is larger than a diameter across inflow end I. Inflow end I may be disposed radially inside the outer frame section. Outflow end O may define a lower end or edge of valve support 104. Thus, in a purely conical arrangement, the valve support section 104 may comprise a smoothly decreasing diameter thereacross. The smooth diameter decrease may extend from outflow end O to inflow end I.

Inflow end I may include one or more lobes extending radially outwardly. Such a lobe may interrupt the smooth conical profile. A lobe may be provided for each prosthetic leaflet 106 attached within the inner valve support 104. The lobe may provide a leaflet with more freedom of movement. A lobe may provide leaflets with improved coaptation.

Prosthetic leaflets 106 may be disposed or spaced above the native leaflets when the prosthetic valve stent device 100 is implanted. Prosthetic leaflets 106 may be attached and spaced sufficiently away from (above) the native leaflets so as to not physically interfere or interact with the native leaflets and the resulting blood flow.

The stent cells that transition from the outer section to the inner section of the stent may be referred to as transition cells forming illustrative transition section 108.

The outer and inner sections of the stent may be constructed from one continuous structure or may combine two or more structures. Stent structures may be formed using complementary shaped mandrels. Such mandrels may be used to form one or more of outer section 102, transition section 108, inner valve support 104, lobes L, and any other suitable structures as a single monolithic structure.

The outer support structure may be positioned generally so that it engages with tissue and works to prevent a paravalvular leak (PVL). For example, the outer support structure of the prolapse prevention structure may engage, or be integrated with, the transition section described above to provide a barrier against PVL.

FIG. 10 shows illustrative skirt S. Skirt S may include fabric. Skirt S may include tissue. Skirt S may be disposed along an outer surface of outer frame element 102. Skirt S may be disposed along the outer surface of the transition section 108. Skirt S may be disposed along the inner surface, or inwardly facing surface, of inner valve support 104. Skirt S may face the flow channel defined therein from the inflow end I to the outflow end O.

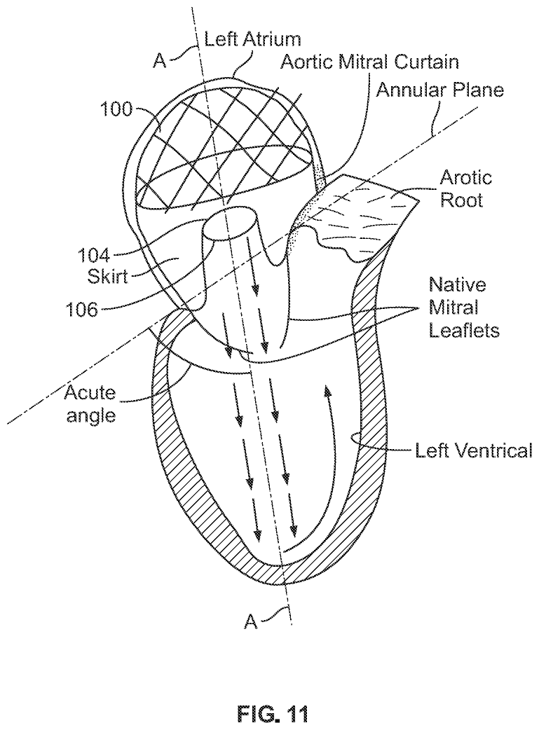

The illustrative devices of FIGS. 9 and 10 are shown implanted in the left atrium in FIG. 11. There, the device 100 is shown as expanded and implanted such that the prosthetic leaflets 106 are spaced above the native mitral leaflets. The expanded device 100 may engage, and may accommodate, the aortic root, enabling the device 100 to expand and orient as adjusted to the aortic root. The expanded device 100 may engage, and may accommodate, the aortic mitral curtain, enabling the device 100 to expand and orient as adjusted to the aortic mitral curtain. The aortic mitral curtain may separate the aortic valve from the mitral valve. This may allow device 100 to provide a flow channel through valve support 104 that is not at a perpendicular angle with the annular plane. Not perpendicular may be referred to alternately herein as oblique. In a manner very much like that of the unmodified, natural blood flow of FIG. 4, device 100 in FIG. 11 may provide a blood flow (represented by axis A) defining an acute angle, in the lateral-medial plane on the posterior side between axis A and the annular plane. Accordingly, as with FIG. 4, the device 100 in FIG. 11 may provide a blood flow impingement that is focused on the posterior lateral side of the left ventricle.

As will now be apparent, the device 100 may be implanted within the left atrium such that the implanted and expanded device 100 is asymmetrically disposed or arranged relative to the annular plane as well as relative to a central axis A running through the flow channel of the valve support 104. As is shown in FIG. 11, the device 100 may comprise the ability to deform and/or expand to accommodate and/or engage anatomical structures and tissue within the left atrium. This capability may enable a tilt in the orientation of the device 100 and in the provision of the acute angle between the resulting blood flow and annular plane as shown in FIG. 11. Central axis A may represent a central axis of the blood flow channel. Central axis A may represent a central axis of the inner valve support. A central axis defined by device 100 may be oblique to central axis A.

Certain structural accommodations may provide a desired tilted orientation of the device relative to the annular plane. This may include providing an asymmetric, or non-uniform, expanded outer section 102. A portion of section 102 may have a larger outer expandable radius than another portion of outer section 102. A portion of outer section 102 may include a stent material that is more (or less) compliant or stiff or flexible than another portion of outer section 102. This may facilitate an asymmetric, or non-uniform, expansion within the left atrium. The transition section 108 may be modified to take on an asymmetric, or non-uniform, expanded configuration in order to achieve the desired orientation of FIG. 11. Moreover, in addition, or in combination with these strategies, valve support 104 may be adapted to be slightly tilted, or asymmetrically disposed, within the expanded and implanted device 100 to ensure that the channel defined within valve support 104 and resulting blood flow therethrough is directed at the desired posterior location within the left ventricle.

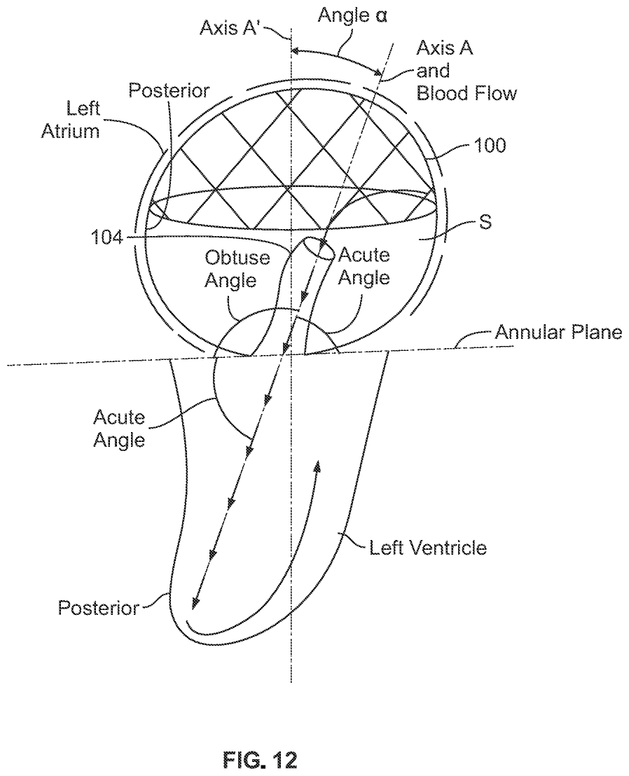

FIG. 12 shows an illustrative structure in which inner valve support 104 may be asymmetrically disposed within the stent frame of device 100. Valve support 104 may extend radially inward from the stent frame at an angle relative to, and spaced away, from a central axis A′ that bisects the otherwise symmetrical stent frame as illustrated. Central axis A′ may be a central axis of device 100. Valve support 104 thus may be arranged with respect to both the stent frame and the annular plane such that the flow channel defined through valve support 104 (as represented generally by axis A) is at a non-zero angle to the central axis A′. Central axis A may represent a central axis of the blood flow channel. Central axis A may represent a central axis of valve support 104. In this manner, valve support 104 may direct blood flow through the defined flow channel toward the posterior lateral portion of the left ventricle. The stent frame, including outer section 102, may be expanded asymmetrically as described above with central axis A′ oblique to the annular plane, or may be symmetrically expanded, so that the central axis A′ is at a 90-degree angle with the annular plane. In either case, however, Axis A of valve support 104 may be angled as shown in FIG. 12 to direct blood flow toward the posterior lateral portion of the left ventricle.

It is noteworthy that the various embodiments of the presently described prosthetic valve stent device 100 may be delivered percutaneously via one of at least the following known access and delivery routes: femoral access, venous access, trans-apical, trans-aortic, trans-septal, and trans-atrial, retrograde from the aorta delivery techniques. Alternatively, the prosthetic valve stent device 100 may be delivered and implanted using surgical and/or open-heart techniques.

The illustrative devices of FIGS. 9, 10 and 12 may be implanted in a right atrium to direct blood flow from the right atrium to the right ventricle along a natural blood flow path. A natural blood flow path in the right side of the heart may be through the tricuspid valve toward a posterior side of the right ventricle. A natural blood flow path in the right side of the heart may be through the tricuspid valve toward a posterior lateral side of the right ventricle.

Apparatus and methods described herein may include the prosthetic heart valve stent for implanting in an atrium of a heart. The atrium may be a right atrium. The atrium may be a left atrium. The stent may include the outer section. The outer section may define a first central axis. The first central axis may extend between a top of the stent and a bottom of the stent. The top of the stent may include a cap.

The outer section, in an unconstrained state, may be spheroidal, exclusive of the bottom portion. The outer section, in an unconstrained state, may define any other suitable shape.

The stent may include the inner valve support section. The inner valve support section may extend upward within the outer section and define a second central axis oblique to the first central axis. The inner valve support section may define a blood flow channel. The second central axis may extend along the blood flow channel circumscribed by the inner valve support.

The stent may include the transition section extending between the outer section and the inner valve support section. The transition section may be positioned at the bottom of the stent.

The first central axis and the second central axis may intersect at an angle of approximately 12 degrees. The first central axis and the second central axis may intersect at an angle in the range of 5-30 degrees. The intersection of the first central axis and the second central axis may be illustrated by Angle α in FIG. 12. First central axis may be axis A′. Second central axis may be axis A.

The inner valve support section may include an inflow end and an outflow end. Blood may move through a blood flow channel defined between the inflow and outflow ends. The inner valve support section may be configured to support prosthetic valve leaflets. The prosthetic valve leaflets may be configured to allow blood flow from the inflow end to the outflow end and prevent blood flow from the outflow end to the inflow end.

When the stent is implanted in the atrium such that a bottom portion of the stent is positioned on an upper annular surface of an annulus, the first central axis may be perpendicular to an annular plane defined by the upper annular surface. The second central axis may be oblique to the annular plane. The annular plane may be an annular plane defined by the upper annular surface. The inner valve support section may direct blood through the annulus toward a posterior wall of the ventricle.

When the stent is implanted in the atrium such that the bottom portion of the stent is positioned on the upper annular surface, the first central axis may be oblique to the annular plane. When the stent is implanted in the atrium, the stent may conform to the anatomy of the atrium, including the aortic mitral curtain, as discussed herein and as described in reference to FIG. 11. This may deform the stent so that the first central axis is oblique to the annular plane.

When the stent is implanted in the atrium such that a bottom portion of the stent is positioned on the upper annular surface, the stent may not extend into the annulus.

The inner valve support section may be configured to direct blood through the annulus toward a posterior lateral wall of the ventricle. The inner valve support section may be configured to direct a flow of blood through the annulus toward a posterior wall of the ventricle. The inner valve support section may be configured to direct a flow of blood through the annulus toward a posterior lateral wall of the ventricle.

Apparatus and methods described herein may include methods for implanting the prosthetic heart valve stent in an atrium of a heart. The methods may include positioning the stent in the atrium in a collapsed state. The stent may include the outer section. The outer section may define the first central axis. The stent may include the inner valve support section. The inner valve support section may be positioned within the outer section and may define the second central axis oblique to the first central axis.

The methods may include expanding the stent such that a bottom of the outer section is positioned on the upper annular surface of the annulus. The methods may include expanding the stent such that the first central axis is perpendicular to the annular plane defined by the upper annular surface. The methods may include expanding the stent such that the second central axis is oblique to the annular plane. The methods may include expanding the stent such that the second central axis intersects a posterior wall of a ventricle of the heart. After the expanding, the second central axis may intersect a posterior lateral wall of a ventricle of the heart.

The methods may include expanding the stent such that the first central axis is oblique to an annular plane defined by the upper annular surface.

The methods may further include expanding the stent so that a top portion of the stent is positioned at a highest point in the atrium.

The inner valve support section, after the expanding, may direct blood through the annulus and towards a posterior wall of the ventricle.

The inner valve support section, after the expanding, may direct blood through the annulus and toward a posterior lateral wall of the ventricle.

The inner valve support section, after the expanding, may direct blood flow through the annulus and towards a posterior wall of the ventricle.

The inner valve support section, after the expanding, may direct blood flow through the annulus and towards a lateral posterior wall of the ventricle.

Apparatus and methods described herein may include a prosthetic heart valve stent for implanting in an atrium of a heart. The atrium may be a right atrium. The atrium may be a left atrium. The stent may include the outer section. The outer section may define an outer section central axis. The outer section central axis may be the first central axis. The stent may include the inner valve support section. The inner valve support section may extend upward within the outer section. The inner valve support section may define a central axis. The central axis of the inner valve support section may be the second central axis. The first central axis may be collinear with the second central axis. The first central axis may be oblique to the second central axis.

When the stent is expanded in the atrium to press against atrial tissue, the outer section may conform to anatomy of the atrium. The anatomy may include the aortic mitral curtain. Conformance of the outer section to the anatomy, such as the aortic mitral curtain, may cause a bottom portion of the outer section to be seated on the annular plane such that the outer section central axis intersects the annular plane at an oblique angle. The annular plane may be defined by the upper annular surface of the annulus.

Conformance of the outer section to the anatomy, such as the aortic mitral curtain, may cause the central axis of the inner valve support section to intersect with a posterior wall of a ventricle of the heart. The posterior wall may be a posterior lateral wall.

Conformance of the outer section to the to the anatomy, such as the aortic mitral curtain, may cause the inner valve support to direct blood flow through the annulus towards a posterior wall of the ventricle of the heart. The posterior wall may be the posterior lateral wall.

The outer section central axis may form an angle of approximately 12 degrees with a normal vector of the annular plane. The outer section central axis may form an angle in the range of 5-30 degrees with a normal vector of the annular plane. The central axis of the inner valve support may form an angle of approximately 12 degrees with a normal vector of the annular plane. The central axis of the inner valve support may form an angle in the range of 5-30 degrees with a normal vector of the annular plane.

A central axis of the inner valve support section may be parallel to the outer section central axis. A central axis of the inner valve support section may be oblique to the outer section central axis.

When the stent is expanded in the atrium to press against atrial tissue, the outer section may conform to the aortic root. Conformance of the stent to the aortic root may affect the positioning of the stent as discussed herein in reference to conformance of the stent to the aortic mitral curtain.

Apparatus and methods described herein may include methods for implanting a prosthetic heart valve stent in an atrium of a heart. The methods may include positioning the stent in the atrium in a collapsed state. The stent may include the outer section defining an outer section central axis and the inner valve support section positioned within the outer section. The methods may include expanding the stent in the atrium such that the outer section conforms to an aortic mitral curtain to cause a bottom portion of the outer section to be seated on the annular plane such that the outer section central axis is oblique to the annular plane. The annular plane may be defined by an upper annular surface of an annulus.

The prosthetic mitral valve device may be delivered into the atrium by an access route selected from the group consisting of: transapical; transfemoral; transatrial; and transseptal delivery techniques.

Apparatus and methods described herein may include a prosthetic valve device. The prosthetic valve device may be adapted for expansion and implantation into a patient's atrium. The atrium may be a right atrium. The atrium may be a left atrium. When the prosthetic valve device is implanted in the left atrium, it may be referred to as a prosthetic mitral valve device and may be adapted to preserve normal blood flow between the left atrium, the left ventricle and the aorta. When the prosthetic valve device is implanted in the right atrium, it may be adapted to preserve normal blood flow between the right atrium, the right ventricle and the pulmonary artery. Apparatus and methods relating to the prosthetic mitral valve device described herein may be applied to the right atrium.

The device may include a collapsible and expandable stent having an outer section comprising an outer surface, an inner surface, and defining an interior. The expanded stent may include a bisecting central axis. The device may include a valve support defined by the collapsible and expandable stent. The valve support may extend radially upward into the interior of the outer section. The valve support may comprise an inflow end and an outflow end. The inflow end may extend radially upward into the outer section. A blood flow channel may be defined between the inflow and outflow ends. The valve support may be inverted entirely within the interior of the outer section. A central axis may be defined as extending through the blood flow channel. The device may include a plurality of prosthetic valve leaflets disposed within the blood flow channel defined by the valve support section. The prosthetic valve leaflets may be configured to allow flow from the inflow end to the outflow end of the flow channel and prevent flow from the outflow end of the flow channel to the inflow end of the flow channel. A central axis extending through the blood flow channel may be offset from the bisecting central axis of the expanded stent by an angle.

Apparatus and methods described herein may include methods for preserving a patient's normal blood flow after implanting a prosthetic mitral valve in the patient's left atrium. The methods may include providing and implanting a prosthetic mitral valve device within the patient's left atrium. The implanted prosthetic mitral valve device may include a collapsible and expandable stent. The stent may include a valve support having prosthetic leaflets operationally disposed within the valve support for one-way flow through a blood flow channel defined by the valve support. A central axis may be defined through the blood flow channel. The methods may include ensuring that a portion of the collapsible and expandable stent engages an annular surface along an annular plane defined within the left atrium. The collapsible and expandable stent may be deformed by engaging the aortic root. The collapsible and expandable stent may be deformed by engaging the aortic mitral curtain. The central axis may cross the annular plane at a perpendicular angle. The central axis may cross the annular plane at an oblique angle.

The methods may include ensuring that the implanted prosthetic mitral valve device does not interfere with the native mitral leaflets. The methods may include ensuring that the blood channel defined by the valve support is disposed above the native mitral leaflets. This may ensure that the blood flow channel targets blood flow onto the native mitral valve leaflets. This may ensure that the blood flow channel deposits blood flowing through the blood flow channel onto the native mitral valve leaflets. Subsequent downstream flow may be directed at a posterior lateral side of the left ventricle. Subsequent downstream blood flow may be directed at a posterior lateral side of the left ventricle.

The methods may include the prosthetic mitral valve device comprising the collapsible and expandable stent further having an outer section comprising an outer surface, an inner surface, and defining an interior. The valve support may extend radially upward into the interior of the outer section and comprise an inflow end and an outflow end. The inflow end may extend radially upward into the outer section. The blood flow channel may be defined between the inflow and outflow ends. The valve support may be inverted entirely within the interior of the outer section. The device the stent may include a plurality of prosthetic valve leaflets disposed within the flow channel defined by the valve support section. The prosthetic valve leaflets may be configured to allow flow from the inflow end to the outflow end of the flow channel and prevent flow from the outflow end of the flow channel to the inflow end of the flow channel. The stent may include a collapsible and expandable transition cell section configured to transition the outer section to the valve support. The valve support may extend radially upward into the interior of the outer section. The transition section may include an outer surface and an inner surface that faces the interior defined by the outer section.

The methods may include the expanded stent including a bisecting central axis. The central axis of the blood flow channel may not be offset from the bisecting central axis by an angle, wherein the blood flow channel directs flow downstream toward the native mitral valve leaflets. The central axis of the blood flow channel may be offset from the bisecting central axis by an angle, wherein the blood flow channel directs flow downstream toward the native mitral valve leaflets.

The prosthetic mitral valve device may be delivered into the atrium by an access route in the group consisting of: transapical; transfemoral; transatrial; and transseptal delivery techniques.

Certain aspects described herein may be readily applicable to single or two chamber solutions, unless otherwise indicated. Moreover, certain aspects discussed herein may be applied to preservation and/or replacement of native valve functionality, with improved native leaflet coaptation and/or prolapsing, and are not, therefore, limited to the mitral valve and may be extended to include devices and methods for treating the tricuspid valve, the aortic valve and/or pulmonary valves.

The description of apparatus and methods set forth herein is illustrative and is not intended to limit the scope of the disclosure. Features of various embodiments may be combined with other embodiments within the contemplation of this disclosure. Variations and modifications of the embodiments disclosed herein are possible, and practical alternatives to and equivalents of the various elements of the embodiments would be understood to those of ordinary skill in the art upon study of this disclosure. These and other variations and modifications of the embodiments disclosed herein may be made without departing from the scope and spirit of the disclosure.

All ranges and parameters disclosed herein shall be understood to encompass any and all subranges subsumed therein, every number between the endpoints, and the endpoints. For example, a stated range of “1 to 11” should be considered to include any and all subranges between (and inclusive of) the minimum value of 1 and the maximum value of 11; that is, all subranges beginning with a minimum value of 1 or more (e.g. 1 to 6.1), and ending with a maximum value of 11 or less (e.g., 2.3 to 10.4, 3 to 8, 4 to 7), and finally to each number 1, 2, 3, 4, 5, 6, 7, 8, 10, and 11 contained within the range.

Thus, apparatus and methods for a prosthetic heart valve that provides a natural blood flow have been provided. Persons skilled in the art will appreciate that the present invention may be practiced by other than the described embodiments, which are presented for purposes of illustration rather than of limitation.

What is claimed is:

1. A prosthetic heart valve stent for implanting in an atrium of a heart, the stent comprising:
an outer section defining a first central axis; and
an inner valve support section extending upward within the outer section and defining a second central axis oblique to the first central axis;
wherein when the stent is implanted in the atrium such that a bottom portion of the stent is positioned on an upper annular surface of an annulus:
the second central axis is oblique to an annular plane defined by the upper annular surface; and
the inner valve support section directs blood through the annulus toward a posterior wall of the ventricle.

2. The stent of claim 1 wherein, when the stent is implanted in the atrium such that the bottom portion of the stent is positioned on the upper annular surface, the first central axis is perpendicular to the annular plane.

3. The stent of claim 1 wherein:
the inner valve support section comprises an inflow end and an outflow end; and
blood moves through a blood flow channel defined between the inflow and outflow ends.

4. The stent of claim 1 wherein, when the stent is implanted in the atrium such that the bottom portion of the stent is positioned on the upper annular surface, the first central axis is oblique to the annular plane.

5. The stent of claim 3 wherein the inner valve support section is configured to support prosthetic valve leaflets that allow blood flow from the inflow end to the outflow end and prevent blood flow from the outflow end to the inflow end.

6. The stent of claim 1 wherein the first central axis and the second central axis intersect at an angle of approximately 12 degrees.

7. The stent of claim 1 wherein the first central axis and the second central axis intersect at an angle in the range of 5-30 degrees.

8. The stent of claim 1 wherein the stent does not extend into the annulus.

9. The stent of claim 1 wherein the inner valve support section is configured to direct blood through the annulus toward a posterior lateral wall of the ventricle.

10. The stent of claim 1 wherein the blood directed by the inner valve support section is a flow of blood.

11. The stent of claim 1 wherein:
the blood directed by the inner valve support section is a flow of blood; and
the flow of blood is directed toward a posterior lateral wall of the ventricle.

12. The stent of claim 1 wherein the first central axis extends between a top of the stent and a bottom of the stent.

13. The stent of claim 12 further comprising a transition section extending between the outer section and the inner valve support section, the transition section being positioned at the bottom of the stent.

14. The stent of claim 1 wherein the second central axis extends along a blood flow channel circumscribed by the inner valve support section.

15. A method for implanting a prosthetic heart valve stent in an atrium of a heart, the method comprising:
positioning the stent in the atrium in a collapsed state, the stent comprising:
an outer section defining a first central axis; and
an inner valve support section positioned within the outer section and defining a second central axis oblique to the first central axis;
expanding the stent such that:
a bottom of the outer section is positioned on an upper annular surface of an annulus;
the second central axis is oblique to an annular plane defined by the upper annular surface; and
the second central axis intersects to a posterior wall of a ventricle of the heart.

16. The method of claim 15 further comprising expanding the stent so that a top portion of the stent is positioned at a highest point in the atrium.

17. The method of claim 15 wherein, after the expanding, the second central axis intersects a posterior lateral wall of a ventricle of the heart.

18. The method of claim 15 wherein the inner valve support section, after the expanding, directs blood through the annulus and towards a posterior wall of the ventricle.

19. The method of claim 15 wherein the inner valve support section, after the expanding, directs blood through the annulus and toward a posterior lateral wall of the ventricle.

20. The method of claim 15 wherein the blood directed by the inner valve support section is a flow of blood.

21. The method of claim 15 wherein:
the blood directed by the inner valve support section is a flow of blood; and
the flow of blood is directed toward a posterior lateral wall of the ventricle.

22. The method of claim 15 further comprising expanding the stent so that the first central axis is perpendicular to the annular plane.

23. The method of claim 15 further comprising expanding the stent so that the first central axis is oblique to the annular plane.
